(19) United States
(12) Patent Application Publication
(10) Pub. No.: US 2006/0018778 A1
(43) Pub. Date: Jan. 26, 2006

(54) Hermetic compressor

(75) Inventor: Cheol Seo, Gwangju (KR)

Correspondence Address: LADAS & PARRY, 26 WEST 61ST STREET, NEW YORK, NY, 10023, US

(73) Assignee: SAMSUNG GWANGJU ELECTRONICS CO., LTD.

(21) Appl. No.: 11/093,315

(22) Filed: Mar. 30, 2005

(30) Foreign Application Priority Data
Jul. 20, 2004 (KR) 2004-56456

(51) Int. Cl. F04B 11/00; A61M 1/00

(52) U.S. Cl. 417/540; 417/437; 417/902

(57) ABSTRACT

A hermetic compressor comprising a discharge muffler. The discharge muffler includes a dented muffler body defined in a cylinder block and a muffler cover to cover the muffler body. The muffler body has a partition to divide the interior of the discharge muffler into a pair of first and second resonance chambers. The partition is integrally formed with the muffler body upon casting of the cylinder block. The partition protrudes from a bottom surface of the muffler body toward the muffler cover so that it is spaced apart from the muffler cover, thereby defining a first communicating portion between the partition and the muffler cover. A refrigerant, introduced into the first resonance chamber, is compressed while passing through the first communicating portion, and is diffused in the second resonance chamber. In this way, the pulsation of the refrigerant is attenuated.

### CROSS-REFERENCE TO RELATED APPLICATION

This application claims the benefit of Korean Patent Application No. 2004-56456, filed on Jul. 20, 2004 in the Korean Intellectual Property Office, the disclosure of which is incorporated herein by reference.

### BACKGROUND OF THE INVENTION

1. Field of the Invention

The present invention relates to a hermetic compressor, and, more particularly, to a hermetic compressor having a discharge muffler capable of attenuating noise generated when a compressed refrigerant is discharged to the outside.

2. Description of the Related Art

In general, a hermetic compressor is designed to suction a refrigerant into a hermetic casing thereof to compress it, and discharge the compressed refrigerant to the outside. Such a hermetic compressor comprises a compressing unit to compress the refrigerant and a driving unit to drive the compressing unit.

The compressing unit is disposed inside the hermetic casing, and includes a cylinder block defining a compression chamber, and a piston reciprocating inside the compression chamber upon receiving power transmitted from the driving unit. A cylinder head is disposed at one side of the cylinder block and is formed with a suction chamber and a discharge chamber, which communicate with the outside.

A valve unit is provided between the cylinder block and the cylinder head in order to permit selective communication between the suction chamber and the compression chamber or between the discharge chamber and the compression chamber.

In order to attenuate noise of the refrigerant discharged from the discharge chamber, a pair of discharge mufflers are provided at both ends of the cylinder block, respectively. A respective one of the discharge mufflers has a muffler body defined in the cylinder block, and a muffler cover to cover the muffler body. A baffle is mounted in the discharge muffler in order to attenuate vortex motion or pulsation of the refrigerant.

However, the conventional hermetic compressor configured as stated above has a problem in that the baffle must be present inside the discharge muffler, resulting in a complicated manufacturing process and high manufacturing cost.

Further, when the baffle is displaced out of its original installation position due to the pressure of the refrigerant, this hinders proper discharge of the refrigerant.

### SUMMARY OF THE INVENTION

The present invention has been made in view of the above mentioned problems, and an aspect of the invention is to provide a hermetic compressor having a discharge muffler which is improved in structure to achieve enhanced productivity and reliability.

In accordance with an aspect, the present invention provides a hermetic compressor comprising a cylinder block defining a compression chamber, a piston reciprocating inside the compression chamber to compress a refrigerant, and a discharge muffler configured to receive the refrigerant compressed in the compression chamber, wherein the discharge muffler includes a muffler body defined in the cylinder block and a muffler cover to cover the muffler body, and wherein the muffler body has an integral partition to divide the interior of the discharge muffler into a pair of resonance chambers communicating with each other.

The partition may be integrally formed with the muffler body by casting.

The partition may protrude from a bottom surface of the muffler body toward the muffler cover so that it is spaced apart from the muffler cover by a predetermined distance.

The partition may be inwardly spaced apart from an inner circumference of the muffler body by a predetermined distance.

One of the resonance chambers may be provided with an inlet to permit the resonance chamber to communicate with the compression chamber, and a discharge tube may be located in the other resonance chamber in order to discharge the refrigerant in the interior of the discharge muffler to the outside.

The muffler cover may be coupled to the muffler body via a bolt, and the partition has a screw bore to permit the bolt to be fastened therethrough.

An auxiliary muffler may be provided in the cylinder block to communicate with the discharge muffler via an auxiliary channel defined in the cylinder block.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more easily appreciated from the following description of the embodiments, taken in conjunction with the accompanying drawings of which:

FIG. 1 is a side sectional view illustrating a hermetic compressor in accordance with the present invention;

FIG. 2 is a plan view illustrating a discharge muffler provided in the hermetic compressor in accordance with a first embodiment of the present invention;

FIG. 3 is a sectional view of the discharge muffler shown in FIG. 2;

FIG. 4 is a cross sectional view taken along line A-A shown in FIG. 3;

FIG. 5 is a sectional view of a discharge muffler in accordance with a second embodiment of the present invention;

FIG. 6 is a cross sectional view taken along line B-B shown in FIG. 5; and

FIG. 7 is a sectional view illustrating an auxiliary muffler provided in a cylinder block in addition to the discharge muffler in accordance with the present invention.

### DETAILED DESCRIPTION OF THE PREFERRED EMBODIMENTS

Reference will now be made in detail to the embodiments of the present invention, examples of which are illustrated in the accompanying drawings, wherein like reference numerals refer to the like elements throughout.

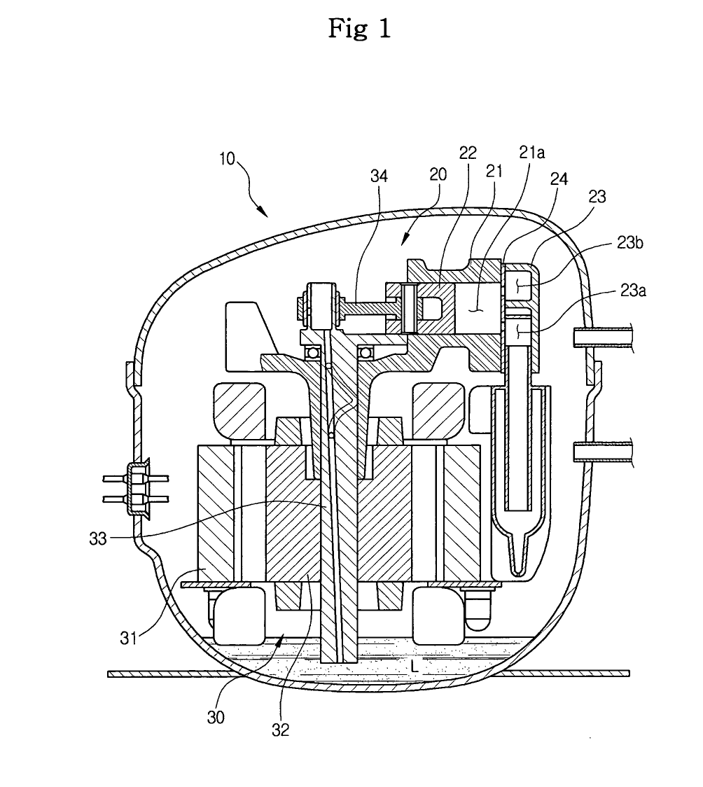

FIG. 1 is a side sectional view of a hermetic compressor in accordance with the present invention. Referring to FIG. 1, the hermetic compressor of the present invention comprises a compressing unit 20 disposed inside a hermetic casing 10 to compress a refrigerant, and a driving unit 30 to generate power required to drive the compressing unit 20.

The compressing unit 20 includes a cylinder block 21 internally defining a compression chamber 21a, and a piston 22 reciprocating inside the compression chamber 21a so as to suction, compress and discharge the refrigerant. A cylinder head 23 is disposed at one end of the cylinder block 21 to face each other. The cylinder head 23 internally defines a suction chamber 23a and a discharge chamber 23b. A valve unit 24 is interposed between the cylinder block 21 and the cylinder head 23 so as to selectively communicate the suction chamber 23a or the discharge chamber 23b with the compression chamber 21a.

The driving unit 30 operates to reciprocate the piston 22, thereby permitting the refrigerant to be compressed inside the compressing unit 20. The driving unit 30 includes a stator 31 fixedly disposed inside the hermetic casing 10, and a rotor 32 inwardly spaced apart from the stator 31 to electromagnetically interact with the stator 31. A rotating shaft 33 is coupled in the center of the rotor 32 to rotate simultaneously with the rotor 32. An end of the rotating shaft 33 is coupled to one end of a connecting rod 34 to permit rotation of the connecting rod 34. The rotation of the connecting rod 34 causes the other end thereof to linearly reciprocate to thereby move the piston 22 forward or backward.

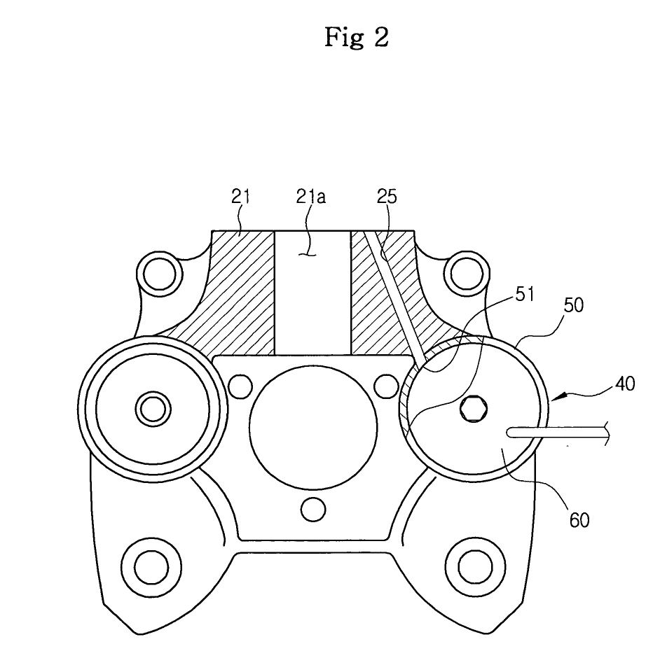

FIG. 2 is a plan view illustrating a discharge muffler provided in the hermetic compressor in accordance with a first embodiment of the present invention. Referring to FIG. 2, the discharge muffler 40 according to the first embodiment of the present invention is provided at at least one of both ends of the cylinder block 21 and is adapted to attenuate noise of the refrigerant being discharged out of the compressor. The discharge muffler 40 has a dented muffler body 50 defined in the cylinder block 21 and a muffler cover 60 to cover the muffler body 50.

The muffler body 50 has an inlet 51 to permit introduction of the refrigerant into the discharge muffler 40. The inlet 51 communicates with a guide channel 25 which is perforated through the cylinder block 21 to guide the refrigerant from the compression chamber 21a into the discharge muffler 40.

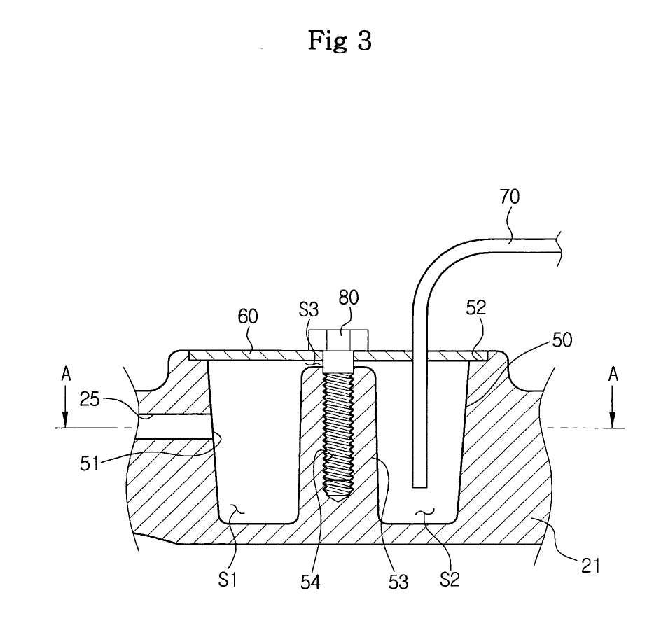

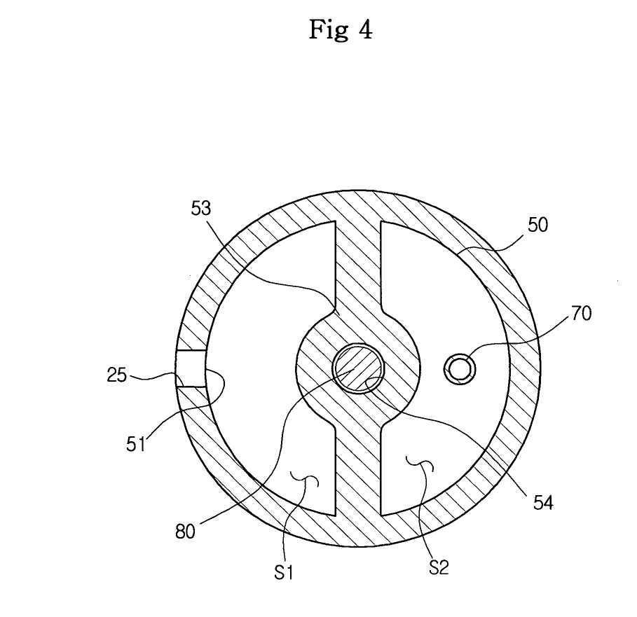

FIG. 3 is a sectional view of the discharge muffler 40 shown in FIG. 2. FIG. 4 is a cross sectional view taken along line A-A shown in FIG. 3. Referring to FIGS. 3 and 4, a seating groove 52 is defined at an upper en of the muffler body 50 for the seating of the muffler cover 60. When the muffler cover 60 covers the muffler body 50, it defines a space to attenuate pulsation of the refrigerant. A discharge tube 70 is mounted in the muffler cover 60 to communicate with the interior of the discharge muffler 40. The discharge tube 70 serves to discharge the refrigerant out of the compressor.

The muffler body 50 has a partition 53 to divide the interior of the discharge muffler 40 into first and second resonance chambers S1 and S2. The partition 53 is integrally formed with the muffler body 50 upon casting of the cylinder block 21.

The partition 53 protrudes from a bottom surface of the muffler body 50 toward the muffler cover 60 so that a distal end thereof is spaced apart from the muffler cover 60 to define a first communicating portion S3 between the partition 53 and the muffler cover 60. That is, the interior of the discharge muffler 40 is divided into the first and second resonance chambers S1 and S2 by the partition 53, and both the first and second resonance chambers S1 and S2 communicate with each other via the first communicating portion S3.

In this case, the inlet 51 is formed at the first resonance chamber S1, and the discharge tube 70 is positioned in the second resonance chamber S2. With such a configuration, the refrigerant, introduced into the first resonance chamber S1, is compressed while passing through the first communicating portion S3, and is diffused in the second resonance chamber S2, thereby being attenuated in pulsation thereof.

Meanwhile, the muffler cover 60 is coupled to the muffler body 50 by means of a bolt 80. The bolt 80 is fastened into a screw bore 54 defined in the partition 53 of the muffler body 50.

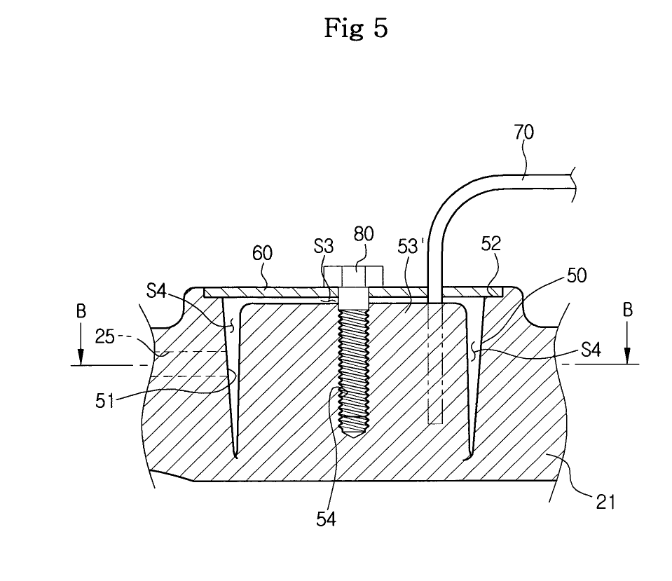

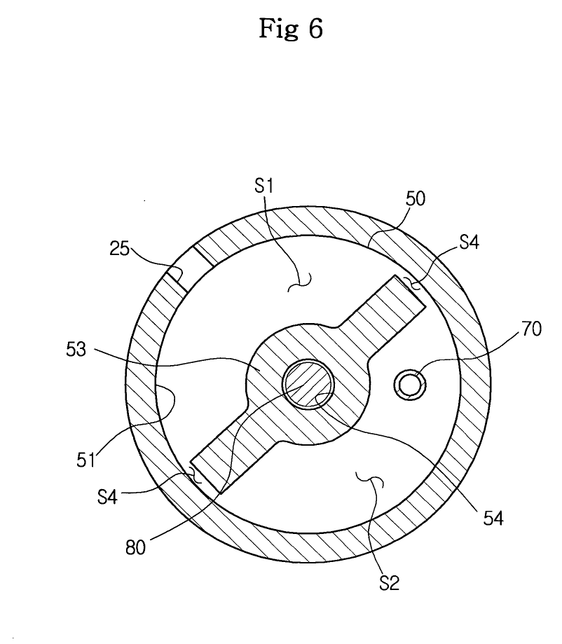

FIG. 5 is a sectional view of a discharge muffler in accordance with a second embodiment of the present invention. FIG. 6 is a cross sectional view taken along line B-B shown in FIG. 5. The second embodiment is identical to the first embodiment except for a partition 53′.

Referring to FIGS. 5 and 6, the partition 53′ is formed at the muffler body 50 so that it is inwardly spaced apart from the muffler body 50. Thereby, in addition to the first communicating portion S3 defined between the partition 53′ and the muffler cover 60, the discharge muffler according to the present embodiment further includes a second communicating portion S4 defined between opposite ends of the partition 53′ and an inner circumference of the muffler body 50.

With such a configuration, the refrigerant flows from the first resonance chamber S1 into the second resonance chamber S2 via both the first communicating portion S3 and the second communicating portion S4.

It will be easily understood that the configuration of the partition provided in the discharge muffler of the present invention is not limited to that of the above described partitions 53 and 53′, and may have various different configurations depending on the characteristics of the compressors.

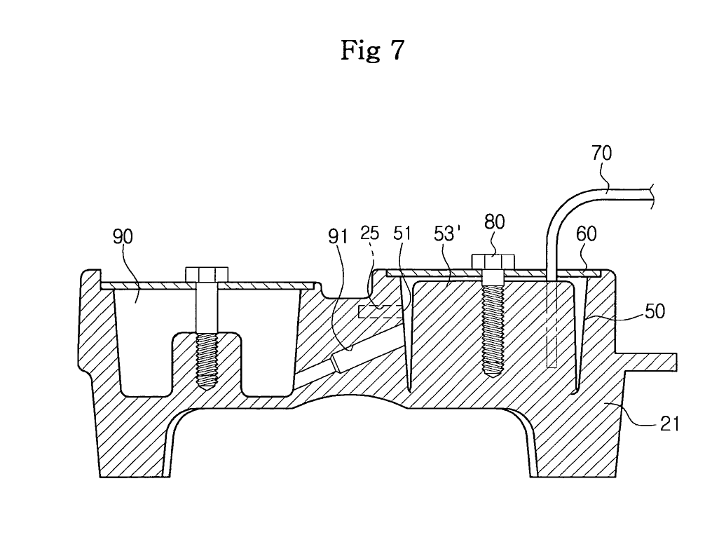

Meanwhile, as shown in FIG. 7, an auxiliary muffler 90 may be provided in the cylinder block 21 in addition to the discharge muffler 40. The auxiliary muffler 90 communicates with the discharge muffler 40 via an auxiliary channel 91 defined in the cylinder block 21. Such an auxiliary muffler 90 more attenuates the pulsation noise of the refrigerant via a Helmholtz resonance chamber defined therein.

Now, the general operation of the hermetic compressor according to the present invention will be explained.

When power is applied to the driving unit 30, the rotor 32 rotates simultaneously with the rotating shaft 33. The rotation of the rotating shaft 33 causes the piston 22 to reciprocate inside the compression chamber 21a to thereby allow the refrigerant to be suctioned from the suction chamber 23a of the cylinder head 23 into the compression chamber 21a and be compressed in the compression chamber 21a. After that, the compressed refrigerant is discharged into the discharge chamber 23b of the cylinder head 23 via the valve unit 24 according to reciprocation of the piston 22.

After that, the refrigerant discharged into the discharge chamber 23b is guided toward the discharge muffler via the guide channel 25 defined in the cylinder block 21. Thereby, the refrigerant, introduced into the discharge muffler via the inlet 51 of the muffler body 50, is attenuated in noise in the first resonance chamber S1. In succession, the refrigerant is compressed while passing through the partition 53, and is diffused in the second resonance chamber S2. In this way, the pulsation of the refrigerant is attenuated. Finally, the refrigerant is discharged out of the compressor via the discharge tube 70 located in the second resonance chamber S2.

As is apparent from the above description, the present invention provides a hermetic compressor having a discharge muffler in which a partition is integrally formed with a muffler body defined in a cylinder block.

Such a configuration permits a refrigerant, introduced into the discharge muffler, to undergo attenuation of pulsation thereof while passing through the partition, enabling effective attenuation of noise of the refrigerant being discharged out of the compressor.

Further, according to the present invention, the partition is integrally formed with the muffler body upon casting of the discharge muffler. This simplifies the manufacturing process of the discharge muffler, reducing the manufacturing cost thereof.

Although an embodiment of the present invention has been shown and described, it would be appreciated by those skilled in the art that changes may be made in this embodiment without departing from the principles and spirit of the invention, the scope of which is defined in the claims and their equivalents.

What is claimed is:

1. A hermetic compressor comprising a cylinder block defining a compression chamber, a piston reciprocating inside the compression chamber to compress a refrigerant, and a discharge muffler configured to receive the refrigerant compressed in the compression chamber,
wherein the discharge muffler includes a muffler body defined in the cylinder block and a muffler cover to cover the muffler body,
wherein the muffler body has an integral partition to divide the interior of the discharge muffler into a pair of resonance chambers communicating with each other.

2. The compressor according to claim 1, wherein the partition is integrally formed with the muffler body by casting.

3. The compressor according to claim 1, wherein the partition protrudes from a bottom surface of the muffler body toward the muffler cover so that it is spaced apart from the muffler cover by a predetermined distance.

4. The compressor according to claim 1, wherein the partition is inwardly spaced apart from an inner circumference of the muffler body by a predetermined distance.

5. The compressor according to claim 1, wherein one of the resonance chambers is provided with an inlet to permit the resonance chamber to communicate with the compression chamber, and a discharge tube is located in the other resonance chamber in order to discharge the refrigerant in the interior of the discharge muffler to the outside.

6. The compressor according to claim 1, wherein the muffler cover is coupled to the muffler body via a bolt, and the partition has a screw bore to permit the bolt to be fastened therethrough.

7. The compressor according to claim 1, wherein an auxiliary muffler is provided in the cylinder block to communicate with the discharge muffler via an auxiliary channel defined in the cylinder block.
